(19) United States
(12) Patent Application Publication
(10) Pub. No.: US 2024/0257980 A1
(43) Pub. Date: Aug. 1, 2024

(54) RECIPIENT SURVIVAL AFTER ORGAN TRANSPLANTATION

(71) Applicant: UNIVERSITY OF SOUTH FLORIDA, Tampa, FL (US)

(72) Inventors: Paul KUO, Tampa, FL (US); Haroon JANJUA, Tampa, FL (US); Michael ROGERS, Tampa, FL (US)

(21) Appl. No.: 18/427,739

(22) Filed: Jan. 30, 2024

Related U.S. Application Data
(60) Provisional application No. 63/482,074, filed on Jan. 30, 2023.

(51) Int. Cl. G16H 50/30; G16H 50/20

(52) U.S. Cl. CPC G16H 50/30; G16H 50/20

(57) ABSTRACT

Methods and systems for predicting recipient survival after an organ transplant are disclosed. The methods and systems include: obtaining a pre-operative organ transplant machine learning model; receiving a donor dataset corresponding to a plurality of factors relating to a given donor; receiving a pre-operative recipient dataset corresponding to a plurality of pre-operative recipient factors; applying the donor dataset and the pre-operative recipient dataset to the pre-operative organ transplant machine learning model; providing a result based on the trained organ transplant machine learning model; receiving a post-operative recipient dataset corresponding to a plurality of post-operative factors, the plurality of post-operative factors relating to a transplantation operation of the patient; determining if the patient exhibits graft dysfunction; applying the pre-operative recipient and the post-operative recipient datasets to a post-operative organ transplant machine learning model; and determining a survival probability. Other aspects, embodiments, and features are also claimed and described.

### CROSS-REFERENCE TO RELATED APPLICATION(S)

This application claims the benefit of U.S. Provisional Patent Application Ser. No. 63/482,074, filed Jan. 30, 2023.

### STATEMENT REGARDING FEDERALLY SPONSORED RESEARCH N/A

### BACKGROUND

Solid organ transplant remains a lifesaving therapy for end-stage organ disease. Despite significant strides in surgical techniques and perioperative management, long-term survival is beleaguered by risks of graft failure, infection, and cancer. The need for precise and personalized survival predictions is paramount, as it can profoundly influence clinical decision-making, potentially improving post-transplant survival outcomes.

Numerous studies have established risk modeling strategies for organ transplants. These models have identified prognostic factors related to donor and recipient characteristics, surgical procedures, complications, biomarkers, and radiology. While these models are supported by clinical validation, their foundation lies in traditional statistical approaches, assuming a linear and cumulative interaction of variables influencing patient survival. Recent studies indicate that the interconnection of clinical variables in specific outcomes may not be sufficiently understood using traditional methods. The importance of variables may fluctuate, depending on the presence or absence of other accompanying features.

Furthermore, retrospective datasets that are used for traditional or linear statistical studies are often homogenous and include multiple data items that may not be available for a given patient/donor pair, or which may not yet be available (or may be subject to change). Furthermore, previous attempts at statistical analyses of organ transplant survival did not selectively filter out incomplete or inapplicable records, while retaining other outlier cases, and thus may not be personalized to any given subgroup across an entire population.

To overcome these limitations, the present disclosure describes various processes and embodiments that leverage machine learning (ML) tools, as well as specific training and model generation techniques, to provide robust options for generating more personalized organ transplant outcome predictions that can be adaptable to ongoing care and new factors.

### SUMMARY

The following presents a simplified summary of one or more aspects of the present disclosure, to provide a basic understanding of such aspects. This summary is not an extensive overview of all contemplated features of the disclosure and is intended neither to identify key or critical elements of all aspects of the disclosure nor to delineate the scope of any of all aspects of the disclosure. Its sole purpose is to present some concepts of one or more aspects of the disclosure in a simplified form as a prelude to the more detailed description that is presented later.

In some aspects, the present disclosure can provide a method for predicting recipient survival after organ transplant. A pre-operative transplant machine learning model can be obtained. A donor dataset corresponding to a plurality of factors relating to a given donor can be received. A pre-operative recipient dataset corresponding to a plurality of pre-operative recipient factors can be received. The donor dataset and the pre-operative recipient dataset can be applied to the pre-operative organ transplant machine learning model. A result for a patient can be provided based on the trained organ transplant machine learning model. A post-operative recipient dataset corresponding to a plurality of post-operative factors can be received. The plurality of post-operative factors can relate to a transplantation operation of the patient. It may be determined if the patient exhibits graft dysfunction. The pre-operative recipient dataset and the post-operative recipient dataset can be applied to a post-operative organ transplant machine learning model. The survival probability of the patient can be determined.

In further aspects, the present disclosure can provide a method for recipient survival after organ transplant prediction model training. A first training dataset relating to a plurality organ recipients can be received. The first training dataset can include pre-operative and post-operative factors. A second training dataset relating to a plurality of organ donors can be received. The plurality of organ donors can correspond to the plurality of organ recipients. The first training dataset can be filtered to remove post-operative factors, data records for inapplicable recipient treatments, and data records for recipients with graft dysfunction to generate a recipient pre-operative training dataset. A pre-operative machine model can be trained based on the recipient pre-operative training dataset and the second training dataset. The first training dataset can be filtered to remove inapplicable recipient treatments, and recipients with graft dysfunction to generate a recipient post-operative training dataset. A post-operative machine learning model can be trained based on the post-operative training dataset and the second training dataset. The post-operative machine learning model can correspond to the pre-operative machine learning model.

In further aspects, the present disclosure can provide a system for recipient survival after organ transplant prediction. The system can include a memory and a processor communicatively coupled to the memory. The memory can store a set of instructions which, when executed by the processor, can cause the process to obtain a pre-operative organ transplant machine learning model. A donor dataset corresponding to a plurality of factors relating to a given donor can be received. A pre-operative recipient dataset corresponding to a plurality of pre-operative recipient factors can be received. The donor dataset and the pre-operative recipient dataset can be applied to the pre-operative organ transplant machine learning model. A result for a patient can be provided based on the trained organ transplant machine learning model. A post-operative recipient dataset corresponding to a plurality of post-operative factors can be received. The plurality of post-operative factors can relate to a transplantation operation of the patient. It can be determined if the patient exhibits graft dysfunction. The pre-operative recipient dataset and the post-operative recipient dataset can be applied to the post-operative organ transplant machine learning model and a survival probability for the patient can be determined.

These and other aspects of the disclosure will become more fully understood upon a review of the drawings and the detailed description, which follows. Other aspects, features, and embodiments of the present disclosure will become apparent to those skilled in the art, upon reviewing the following description of specific, example embodiments of the present disclosure in conjunction with the accompanying figures. While features of the present disclosure may be discussed relative to certain embodiments and figures below, all embodiments of the present disclosure can include one or more of the advantageous features discussed herein. In other words, while one or more embodiments may be discussed as having certain advantageous features, one or more of such features may also be used in accordance with the various embodiments of the disclosure discussed herein. Similarly, while example embodiments may be discussed below as devices, systems, or methods embodiments it should be understood that such example embodiments can be implemented in various devices, systems, and methods.

### BRIEF DESCRIPTION OF THE DRAWINGS

The patent or patent application file contains at least one drawing in color. Copies of this patent or patent application publication with color drawings will be provided by the Office upon request and payment of the necessary fee.

FIG. 1 is a block diagram conceptually illustrating a system for a recipient survival after organ transplantation prediction.

FIG. 2 is a flowchart illustrating an example recipient survival after organ transplantation prediction process for a machine learning model.

FIG. 3 is a flowchart illustrating a process for generating a machine learning model.

FIG. 4 is an example graph illustrating a receiver operator characteristic curve for logistic regression modeling on liver transplant data, according to some embodiments.

FIG. 5 is an example graph illustrating several Kaplan-Meier survival curves, according to some embodiments.

FIG. 6 illustrates four example Kaplan-Meier survival curve graphs, according to some embodiments.

FIG. 7 is a CART model depicting an example hierarchical association of prediction factors, according to some embodiments.

FIG. 8 is an example graph illustrating several Kaplan-Meier survival curves with a 95% confidence interval, according to some embodiments.

### DETAILED DESCRIPTION

The detailed description set forth below in connection with the appended drawings is intended as a description of various configurations and is not intended to represent the only configurations in which the subject matter described herein may be practiced. The detailed description includes specific details to provide a thorough understanding of various embodiments of the present disclosure. However, it will be apparent to those skilled in the art that the various features, concepts and embodiments described herein may be implemented and practiced without these specific details. In some instances, well-known structures and components are shown in block diagram form to avoid obscuring such concepts.

Various embodiments, features, and examples of the present disclosure can also be found in the attached appendices, comprising academic articles which describe the inventors' work. These articles support the breadth of and are not limiting of the scope of the present disclosure.

### Example Hardware Optimization System

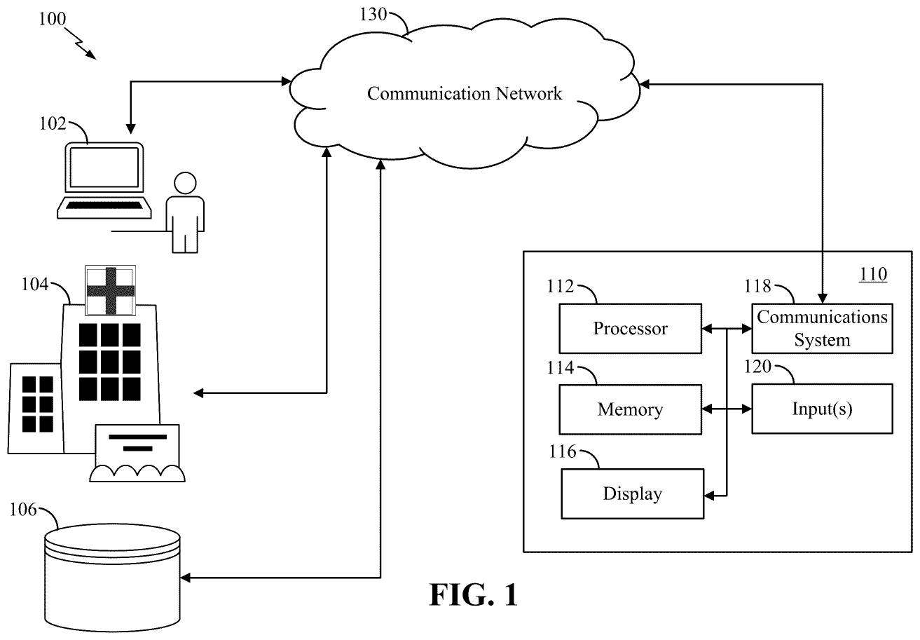

FIG. 1 shows a block diagram illustrating a system for a recipient survival after organ transplantation prediction according to some embodiments. As shown in FIG. 1, computing device 110 can receive multiple pre-operative factor indications (e.g., from a patient device 102, a facility, a clinic, a hospital, or any other suitable data source 106 about a patient), apply entries corresponding to recipient and donor factors to a trained machine learning model, receive a treatment option, and output a survival probability for the patient based on the result for the patient and the treatment option.

In some examples, computing device 110 can include processor 112. In some embodiments, the processor 112 can be any suitable hardware processor or combination of processors, such as a central processing unit (CPU), a graphics processing unit (GPU), an application specific integrated circuit (ASIC), a field-programmable gate array (FPGA), a digital signal processor (DSP), a microcontroller (MCU), etc. Processor 112 may be located within a local (to the user) device (such as a mobile device), may be associated with a system hosting a patient medical record application, may be associated with a system providing information to physicians, may be part of a cloud-based resource, or otherwise, depending on the particular embodiment.

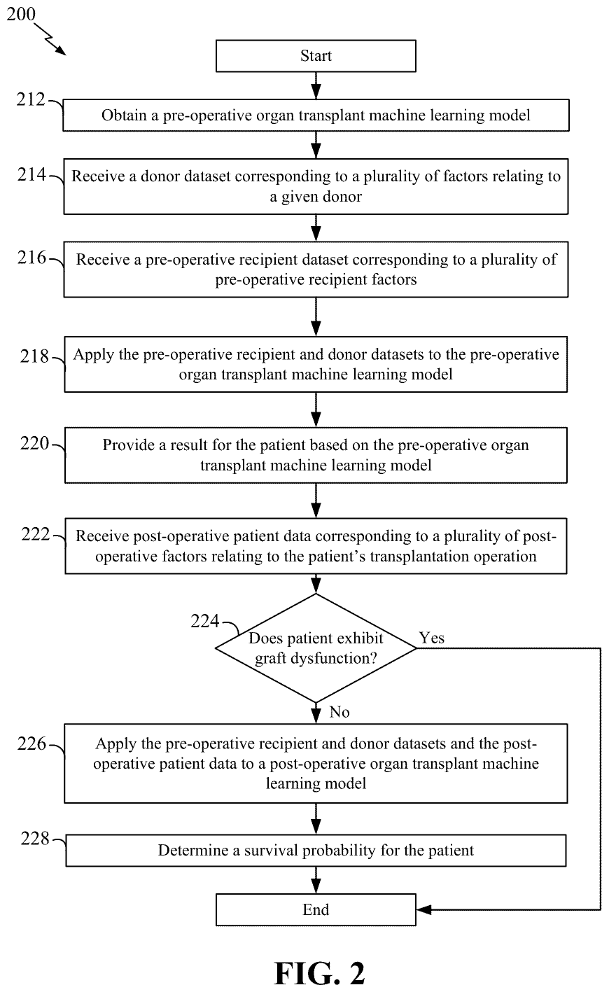

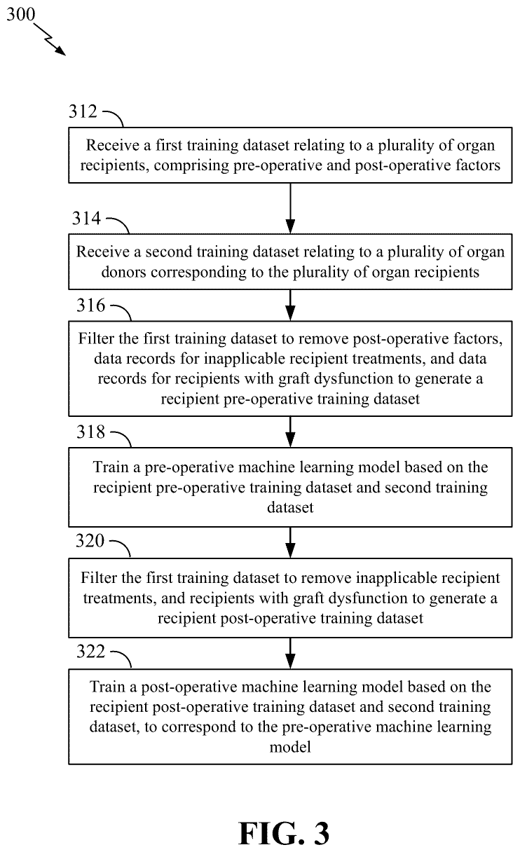

In further examples, computing device 110 can further include a memory 114. The memory 114 can include any suitable storage device or devices that can be used to store suitable data and instructions that can be used, for example, by the processor 112 to receive a first plurality of entries corresponding to a plurality donor factor and a second plurality of entries corresponding to a plurality of recipient factors. The memory 114 can include any suitable volatile memory, non-volatile memory, storage, or any suitable combination thereof. For example, memory 114 can include random access memory (RAM), read-only memory (ROM), electronically-erasable programmable read-only memory (EEPROM), one or more flash drives, one or more hard disks, one or more solid state drives, one or more optical drives, cloud-based resources, etc. In some embodiments, the processor 112 can execute at least a portion of processes 200 and/or 300, described below in connection with FIG. 2 or 3.

In further examples, computing device 110 can further include communications system 118. Communications system 118 can include any suitable hardware, firmware, and/or software for communicating information over communication network 140 and/or any other suitable communication networks. For example, communications system 118 can include one or more transceivers, one or more communication chips and/or chip sets, etc. In a more particular example, communications system 118 can include hardware, firmware and/or software that can be used to establish a Wi-Fi connection, a Bluetooth connection, a cellular connection, an Ethernet connection, a local network, etc.

In further examples, computing device 110 can receive or transmit information (e.g., from or to a patient device 102, a facility, a clinic, a hospital 104, any other suitable data source 106, and/or any other suitable system) over a communication network 130. In some examples, the communication network 130 can be any suitable communication network or combination of communication networks. For example, the communication network 130 can include a Wi-Fi network (which can include one or more wireless routers, one or more switches, etc.), a peer-to-peer network (e.g., a Bluetooth network), a cellular network (e.g., a 3G network, a 4G network, a 5G network, etc., complying with any suitable standard, such as CDMA, GSM, LTE, LTE Advanced, NR, etc.), a wired network, etc. In some embodiments, communication network 130 can be a local area network, a wide area network, a public network (e.g., the Internet), a private or semi-private network (e.g., a corporate or university intranet), any other suitable type of network, or any suitable combination of networks. Communications links shown in FIG. 1 can each be any suitable communications link or combination of communications links, such as wired links, fiber optic links, Wi-Fi links, Bluetooth links, cellular links, etc.

In further examples, computing device 110 can further include a display 116 and/or one or more inputs 120. In some embodiments, the display 116 can include any suitable display devices, such as a computer monitor, a touchscreen, a television, an infotainment screen, etc. to display a report about patient-specific post-surgery mortality prediction, a survival probability of the patient, or any suitable information relating to the patient-specific post-surgery mortality prediction. In further embodiments, the input(s) 120 can include any suitable input devices and/or sensors that can be used to receive user input, such as a keyboard, a mouse, a touchscreen, a microphone, etc.

### Example Process

FIG. 2 is a flow diagram illustrating an example recipient survival after organ transplantation prediction process for a machine learning model. As described below, a particular implementation can omit some or all illustrated features/steps, may be implemented in some embodiments in a different order, and may not require some illustrated features to implement all embodiments. In some examples, an apparatus (e.g., processor 112 with memory 114) in connection with FIG. 1 can be used to perform example process 200. However, it should be appreciated that any suitable apparatus or means for carrying out the operations or features described below may perform process 200.

At step 212, the process 200 obtains a pre-operative organ transplant machine learning model specific to a given type of organ transplant. For example, the model may be specific to a given organ (e.g., liver transplants) and/or to a specific sub-category of transplant (e.g., specific to a transplantation type or procedure). In some examples, the pre-operative organ transplant machine learning model may be trained using datasets of pre-operative records for a plurality of patients and records for those patients' associated organ donors. For example, FIG. 3 (described below in more detail) and the Examples section, below, describe various methods for generating a machine learning model so as to be used in process 200.

At step 214, the process 200 receives a donor dataset corresponding to a plurality of factors relating to a given donor. In some examples, the factors relating to a given donor may include donor age, donor cytomegalovirus status(es) (e.g., based on results of multiple tests over time), and donor pulmonary infection status(es). The factors relating to a given donor may vary based on the type of transplant associated with that donor and the corresponding recipient, as explained in the Examples section below. In further examples, the process 200 may preprocess the donor data to determine if there are any missing fields that would ordinarily be utilized as inputs to the specific pre-operative organ transplant machine learning model from step 212. If any missing fields are identified, process 200 may instead load a machine learning model which was trained or generated on a filtered dataset that lacked the categories of donor factors missing from the donor data obtained in step 214. In this regard, process 200 may depart from traditional statistical analysis notions given the specific domain in which process 200 is intended to be used: for the types of applications in which process 200 is useful, donor data cannot simply be discarded because it lacks relevant factors, given the importance and urgency of transplantation procedures and the scarcity of available donor organs. Moreover, in most cases the donors themselves are no longer living or otherwise in dire health situations that make further data gathering from them or their families or care teams unlikely or unsuitable. Thus, process 200 may instead adapt itself to the existing donor data.

At step 216, the process 200 receives a pre-operative recipient dataset corresponding to a plurality of pre-operative recipient factors. In some examples, the pre-operative recipient factors may include recipient primary payment method, recipient Hepatitis C status(es), recipient diabetes status(es), and/or a plurality of recipient functional statuses before the transplant. The pre-operative recipient factors may vary based on the type of organ, organ transplant, or transplant procedure, as explained with respect to FIG. 3 and the Examples section below. If any factors are identified as missing, a notification may be sent to a user to indicate that said factors are missing data and/or information. Here, however, embodiments of process 200 may require the recipient information be gathered, rather than resorting to a backup model.

At step 218, the process 200 applies the pre-operative recipient and donor datasets to a pre-operative organ transplant machine learning model. In some examples, the pre-operative and donor datasets may be processed using a cross-population machine learning model, such as a machine learning model that was created using one or more survival tree algorithms, such as LongCART. FIG. 3 and the Examples section further describe the generation of such models.

At step 220, the process 200 provides a result for the patient based on the pre-operative organ transplant machine learning model. For example, the result may include a percentage indicating a probability of survival and/or a mortality probability at set intervals of time post-transplant (e.g., 6 months, 1 year, 2 years, 3 years, 5 years, 10 years, etc.). In other examples, the result may be a survival curve with confidence intervals that provide outcome information at a more comprehensive timeline. In some examples, a subgroup into which the recipient falls, and the factors associated with the subgroup, may be indicated in the result. And, any such outputs may also provide an identification of significant factors that influence a recipient's survival predictions, or only factors that can be altered (e.g., BMI), only factors that cannot be altered (e.g., age or donor-specific factors), to allow for the recipient to make more informed choices. In some examples, the result may not account for any data obtained after the operative is performed (i.e., post-operative patient data), as well as any data missing or non-collected.

At step 220, the output of the pre-operative organ transplant machine learning model may be transmitted to a physician treating the recipient, or another member of the care team, to guide patient counseling. In other embodiments, the process 200 may operate outside of the healthcare environment and provide output directly to a patient. And, in yet other embodiments, the outcome information may be provided to an organ donor matching system (e.g., The Organ Procurement and Transplantation Network (OPTN), or healthcare system-specific organ matching system) or to an insurance company or other payor. Thus, output of the model may help inform individuals involved in the recipient's care outside of the primary patient-physician relationship.

In some embodiments, where there are no relevant post-operative factors, the process 200 may end at step 220.

At step 222, the process 200 receives post-operative patient data corresponding to a plurality of post-operative factors relating to the patient's transplantation operation. In some examples, the post operative patient data may include the patient's reintubation status after transplantation, the type of transplant procedure performed on the patient, the patient's ventilator duration post-transplant, and/or the length of the patient stay (i.e., the amount of days between transplantation and discharge). Additionally, if any factors are identified as missing, a notification may be sent to a user to indicate that said factors are missing data and/or information. Again, where such factors are still amenable to gathering and are important to providing the most effective predictors and best information, recipients can be required to provide the associated information.

At step 224, the process 200 determines if the patient exhibits Graft Dysfunction. If the patient does exhibit Graft Dysfunction, the process ends. If the patient does not exhibit Graft Dysfunction, the process proceeds to step 226. In some examples, early Graft failure may be associated with a high mortality rate. The Examples section below further describes the effects and impacts of early Graft failure. Moreover, FIG. 3 and the corresponding description below further explains the reasoning associated with separating Graft Dysfunction as an individual factor. It is notable, however, that the inventors have determined that eliminating one of the most relevant factors (early graft dysfunction) from model training and model input actually helps provide better information in many embodiments.

At step 226, the process 200 applies the pre-operative recipient and donor datasets and the post-operative patient data to a post-operative organ transplant machine learning model. In some examples, the pre-operative datasets and the post-operative data may be processed using a machine learning model which utilizes one or more survival tree algorithms such as LongCART.

At step 228, the process 200 determines a survival probability for the patient. In some examples, the survival probability may be presented as a percentage, and may take a variety of forms such as described above.

### Example Machine Learning Model Training Process

FIG. 3 is a flow diagram illustrating a process for updating a machine learning model. As described below, a particular implementation can omit some or all illustrated features/steps, may be implemented in some embodiments in a different order, and may not require some illustrated features to implement all embodiments. In some examples, an apparatus (e.g., processor 112 with memory 114) in connection with FIG. 1 can be used to perform example process 300. However, it should be appreciated that any suitable apparatus or means for carrying out the operations or features described below may perform process 300.

At step 312, process 300 receives a first training dataset relating to a plurality of organ recipients, comprising pre-operative and post-operative factors. The dataset may include records and fields that are specific to a given organ, organ transplant type, and/or transplantation procedure. In some examples, the first training dataset may include information regarding the age, days on the waiting list, sex, race, ethnicity, body mass index (BMI), previous lung transplant status, and/or insurance of each of the plurality of recipients.

At step 314, process 300 receives a second training dataset relating to a plurality of organ donors corresponding to the plurality of organ recipients. Thus, the information concerning donors will match the specific organ, organ transplant type, and/or transplantation procedure of the first training dataset, because the donors were paired with the recipients of the first training dataset. In some examples, the second training dataset may include information regarding the age, sex, race, ethnicity, BMI, mechanism of death, and/or medical history of each of the plurality of donors. The plurality of organ donors and the corresponding plurality of organ recipients may be split into subgroups using one or more recipient and/or donor factors before proceeding to subsequent steps.

At step 316, process 300 filters the first training dataset to remove post-operative factors, data records for inapplicable recipient treatments, and data records for recipients with Graft Dysfunction to generate a recipient pre-operative training dataset. In some examples, any records with incomplete or missing data may be removed from the training dataset. In some examples, the inventors associated with the present disclosure found that interpolating missing data may generally cause overfitting or improperly impacting outcomes of the model, given the sparsity of available data associated for recipients and their corresponding donors. In some examples, inapplicable treatment options may be removed, such as surgical methods or drugs that are no longer used, as well as treatments not associated with the specific type of transplant associated with the given machine learning model. In further examples, information in certain data fields may be reduced, which unexpectedly results in better accuracies. For example, rather than training a model on actual lab test result values of recipients or donors, such information may be binned so that it merely indicates “high”, “normal,” or “low”. In some examples, however, outliers or unusual circumstances for a given patient and/or donor may still be included in the model because a survival tree model account for specific subgroups and may not present false results for other subgroups. In some embodiments, however, all patients that exhibited early graft failure may be removed from the records.

At step 318, process 300 trains a pre-operative machine learning model based on the recipient pre-operative training dataset and the second training dataset. The pre-operative machine learning model may be utilized in steps 212, 218, and 220 of process 200. In some examples, the pre-operative machine learning model can utilize a survival tree algorithm. For example, a survival tree algorithm may return significant factors and interaction between factors associated with variations in survival rates among the datasets.

At step 320, process 300 filters the first training dataset to remove inapplicable recipient treatments, and recipients with graft dysfunction to generate a recipient post-operative training dataset, etc., just as with step 316. In some examples, patients with graft dysfunction may be excluded from training due to a high mortality rate associated with early graft failure. In other examples. Inapplicable data may include any factors or inputs that are incomplete or missing.

At step 322, the process 300 trains a post-operative machine learning model based on the recipient post-operative training dataset and the second training dataset, to correspond to the pre-operative machine learning model. The post-operative machine learning model may be utilized in step 226 of process 200. In some examples, the post-operative machine learning model can utilize a survival tree algorithm.

### Example #1

Material and Methods: The United Network Organ Sharing (UNOS) Standard Transplant Analysis and Research (STAR) database was queried for all single and double lung transplants in adult recipients (≥18 years old) between 2000 and 2021. The UNOS STAR files contain national patient-level information regarding transplant recipients, deceased and living donors, and waitlist candidates reported to the Organ Procurement and Transplant Network from all transplant centers since October 1987.

Exploratory data analysis was undertaken via visual exploration to identify missing values, as well as frequency/percentage for categorical variables and distribution for numeric variables. Variables with >30% missing values, as well as variables with >5% missing values that did not demonstrate a statistically significant effect on mortality were excluded. These variables were dropped from the dataset to preserve the sample size. Patients were then excluded if any remaining variables were missing from their record (e.g., patient status [alive vs. dead]). Notably, patients with graft survival times less than seven days were excluded as the goal of this study was to predict survival in patients with successful transplant. Additionally, early graft failure is associated with exceedingly high mortality in the literature and 92% mortality in our data. This limits the ability of predictive modeling to determine other influential factors, and thus early graft failure was excluded. Additionally, patients with a concomitant transplant (i.e., heart-lung) were excluded.

Unadjusted bivariate analysis was performed with the resulting patient cohort. T-test and Chi-square tests were performed for numeric and categorical variables, respectively, to determine statistical significance. A p<0.05 was considered significant.

Donor and recipient factors were evaluated with multivariable logistic regression with backward elimination and 10-fold cross validation to identify significant variables. Variables with excessive multicollinearity (variance inflation factor >5) were excluded. A final logistic regression model was created.

After defining the model variables, continuous variables (e.g., days of recipient ventilator support post-transplant) were converted to categorical variables. Data were split into training and testing cohorts of 70% and 30%, respectively in preparation for survival tree modeling. The survival tree was created using the LongCART (‘LongCART’ package, version 3.2) in R, to provide an interpretable prediction of mortality utilizing the resulting variables. The LongCART package, an adapted algorithm of classification and regression tree modeling, constructs a tree for continuous longitudinal data and survival data using baseline covariates as partitioning variables. Tree pruning and model selection was performed based on Akaike information criterion (AIC) and log-likelihood values. Kaplan-Meier plots were produced for each survival tree subgroup.

Data preprocessing was performed using Stata software version 16 (StataCorp, College Station, TX). Modeling and survival probability calculations were performed in R Studio Statistical Analysis software (R Core Team, 2021). This study was deemed exempt from the Institutional Review Board given the deidentified retrospective database nature of this analysis.

Results—Study Population: There were 37,828 adult lung transplant recipients in the dataset. After excluding patients with missing data (n=10,226) and patients with graft survival time of less than seven days (n=306), a total of 27,296 lung transplant patients were included for analysis. Resulting years represented were 2004-2021, with only 5,284 patients transplanted prior to 2010. The recipient's median age was 60 years old (IQR: 51-65), and more than half were male (60.4%). The recipients spent a median of 56 days (IQR: 16-178) on the transplant waiting list (Table 1). Two-third of the donors had positive anti-cytomegalovirus (CMV) antibodies (62.5%). Most of the recipients had restrictive lung disease (57.9%) and received bilateral lung transplant (70.1%).

The deceased patients more often required reintubation (Alive: 14.8% vs Deceased: 21.1%; p<0.001), had longer ventilator support (Alive: 16.8% vs Deceased: 22.1%; p<0.001), and had longer length of stay (LOS) after transplant (Alive: 16 days vs Deceased: 17 days; p<0.001; Table 2).

TABLE 1
Donor and Recipient Baseline Lung Transplant Patient Demographics

| | Total | | Alive | | Deceased | | |
|---|---|---|---|---|---|---|---|
| | (n = 27,296) | | (n = 14,835) | | (n = 12,461) | | p-value |
| Donor | | | | | | | |
| Donor age, y, median (IQR) | 33 | (22-46) | 32 | (23-45) | 33 | (22-47) | 0.037 |
| Pediatric donor | 2,272 | (8.3) | 1,162 | (7.8) | 1,110 | (8.9) | 0.001 |
| Donor sex | | | | | | | 0.40 |
| Female | 10,798 | (39.6) | 5,835 | (39.3) | 4,963 | (39.8) | |
| Male | 16,498 | (60.4) | 9,000 | (60.7) | 7,498 | (60.2) | |
| Donor race | | | | | | | <0.001 |
| White | 16,782 | (61.5) | 9,315 | (62.8) | 7,467 | (59.9) | |
| Black | 4,981 | (18.2) | 2,448 | (16.5) | 2,533 | (20.3) | |
| Hispanic | 4,341 | (15.9) | 2,402 | (16.2) | 1,939 | (15.6) | |
| Asian | 766 | (2.8) | 434 | (2.9) | 332 | (2.7) | |
| American Indian | 133 | (0.5) | 74 | (0.5) | 59 | (0.5) | |
| Pacific Islander | 62 | (0.2) | 34 | (0.2) | 28 | (0.2) | |
| Multiracial | 231 | (0.8) | 128 | (0.9) | 103 | (0.8) | |
| Donor BMI | | | | | | | <0.001 |
| Normal | 11,516 | (42.2) | 6,116 | (41.2) | 5,400 | (43.3) | |
| Underweight | 958 | (3.5) | 525 | (3.5) | 433 | (3.5) | |
| Overweight | 9,199 | (33.7) | 4,986 | (33.6) | 4,213 | (33.8) | |
| Obese | 5,623 | (20.6) | 3,208 | (21.6) | 2,415 | (19.4) | |
| Recipient | | | | | | | |
| Recipient age, y, median (IQR) | 60 | (51-65) | 59 | (50-65) | 61 | (53-66) | <0.001 |
| Total days on waiting list, median | 56 | (16-178) | 51 | (16-163) | 61 | (18-197) | <0.001 |
| (IQR) | | | | | | | |
| Recipient sex | | | | | | | 0.003 |
| Female | 11,000 | (40.3) | 6,100 | (41.1) | 4,900 | (39.3) | |
| Male | 16,296 | (59.7) | 8,735 | (58.9) | 7,561 | (60.7) | |
| Recipient race | | | | | | | <0.001 |
| White | 22,080 | (80.9) | 11,685 | (78.8) | 10,395 | (83.4) | |
| Black | 2,412 | (8.8) | 1,339 | (9.0) | 1,073 | (8.6) | |
| Hispanic | 2,053 | (7.5) | 1,332 | (9.0) | 721 | (5.8) | |
| Asian | 545 | (2.0) | 361 | (2.4) | 184 | (1.5) | |
| American Indian | 94 | (0.3) | 56 | (0.4) | 38 | (0.3) | |
| Pacific Islander | 20 | (0.1) | 13 | (0.1) | 7 | (0.1) | |
| Multiracial | 92 | (0.3) | 49 | (0.3) | 43 | (0.3) | |
| Ethnicity | | | | | | | <0.001 |
| Hispanic/Latino | 2,068 | (7.6) | 1,341 | (9.0) | 727 | (5.8) | |
| Not Hispanic/Latino | 25,228 | (92.4) | 13,494 | (91.0) | 11,734 | (94.2) | |
| Recipient BMI | | | | | | | <0.001 |
| Normal | 10,319 | (37.8) | 5,733 | (38.6) | 4,586 | (36.8) | |
| Underweight | 2,011 | (7.4) | 1,013 | (6.8) | 998 | (8.0) | |
| Overweight | 10,473 | (38.4) | 5,687 | (38.3) | 4,786 | (38.4) | |
| Obese | 4,493 | (16.5) | 2,402 | (16.2) | 2,091 | (16.8) | |
| Previous lung transplant | 946 | (3.5) | 372 | (2.5) | 574 | (4.6) | <0.001 |
| Recipient insurance | | | | | | | 0.91 |
| Private | 13,110 | (48.0) | 7,121 | (48.0) | 5,989 | (48.1) | |
| Public | 14,112 | (51.7) | 7,672 | (51.7) | 6,440 | (51.7) | |
| Uninsured | 74 | (0.3) | 42 | (0.3) | 32 | (0.3) | |

All displayed as n (%) unless otherwise specified.
BMI—body mass index.

TABLE 2
Donor and Recipient Lung Transplant Clinical Variables

| | Total | | Alive | | Deceased | | p- |
|---|---|---|---|---|---|---|---|
| | (n = 27,296) | | (n = 14,835) | | (n = 12,461) | | value |
| Donor | | | | | | | |
| Donor mechanism of death | | | | | | | <0.001 |
| Drowning | 1,274 | (4.7) | 756 | (5.1) | 518 | (4.2) | |
| Seizure | 286 | (1.0) | 166 | (1.1) | 120 | (1.0) | |
| Drug overdose | 2,603 | (9.5) | 1,804 | (12.2) | 799 | (6.4) | |
| Cardiac disease | 1,974 | (7.2) | 1,174 | (7.9) | 800 | (6.4) | |
| Gunshot wound | 5,075 | (18.6) | 2,697 | (18.2) | 2,378 | (19.1) | |
| Blunt injury | 6,474 | (23.7) | 3,450 | (23.3) | 3,024 | (24.3) | |
| Cerebral vascular accident | 8,621 | (31.6) | 4,233 | (28.5) | 4,388 | (35.2) | |
| Natural death | 989 | (3.6) | 555 | (3.7) | 434 | (3.5) | |
| Non-heart beating donor | 939 | (3.4) | 642 | (4.3) | 297 | (2.4) | <0.001 |
| Donor meeting expanded donor | 3,036 | (11.1) | 1,506 | (10.2) | 1,530 | (12.3) | <0.001 |
| criteria | | | | | | | |
| Donor chest x-ray | | | | | | | <0.001 |
| Normal | 11,149 | (40.8) | 5,439 | (36.7) | 5,710 | (45.8) | |
| Abnormal | 15,961 | (58.5) | 9,325 | (62.9) | 6,636 | (53.3) | |
| None | 186 | (0.7) | 71 | (0.5) | 115 | (0.9) | |
| Donor anti-CMV | | | | | | | <0.001 |
| Negative | 10,247 | (37.5) | 5,808 | (39.2) | 4,439 | (35.6) | |
| Positive | 17,049 | (62.5) | 9,027 | (60.8) | 8,022 | (64.4) | |
| Donor history of hypertension | 6,334 | (23.2) | 3,313 | (22.3) | 3,021 | (24.2) | <0.001 |
| Donor history of diabetes | 1,975 | (7.2) | 1,074 | (7.2) | 901 | (7.2) | 0.98 |
| Donor with proteinuria | 11,163 | (40.9) | 6,385 | (43.0) | 4,778 | (38.3) | <0.001 |
| Donor creatinine, normal | 19,907 | (72.9) | 10,725 | (72.3) | 9,182 | (73.7) | 0.01 |
| Donor with any clinical infection | 18,457 | (67.6) | 10,755 | (72.5) | 7,702 | (61.8) | <0.001 |
| Donor with pulmonary infection | 16,420 | (60.2) | 9,683 | (65.3) | 6,737 | (54.1) | <0.001 |
| Donor history of cigarettes use | 2,430 | (8.9) | 1,119 | (7.5) | 1,311 | (10.5) | <0.001 |
| Donor history of cocaine use | 4,293 | (15.7) | 2,562 | (17.3) | 1,731 | (13.9) | <0.001 |
| Donor history of other drug use | 11,559 | (42.3) | 6,893 | (46.5) | 4,666 | (37.4) | <0.001 |
| Donor with tattoo | 11,823 | (43.3) | 7,053 | (47.5) | 4,770 | (38.3) | <0.001 |
| Donor given diuretics* | 19,125 | (70.1) | 10,495 | (70.7) | 8,630 | (69.3) | 0.007 |
| Donor given steroids* | 20,360 | (74.6) | 10,771 | (72.6) | 9,589 | (77.0) | <0.001 |
| Donor given inotropic medication* | 12,901 | (47.3) | 6,532 | (44.0) | 6,369 | (51.1) | <0.001 |
| Donor given synthetic anti-diuretic | 4,428 | (16.2) | 2,202 | (14.8) | 2,226 | (17.9) | <0.001 |
| hormone* | | | | | | | |
| Donor given arginine vasopressin* | 17,955 | (65.8) | 9,969 | (67.2) | 7,986 | (64.1) | <0.001 |
| Donor given vasodilators* | 4,595 | (16.8) | 2,624 | (17.7) | 1,971 | (15.8) | <0.001 |
| Donor given antihypertensive(s)* | 8,601 | (31.5) | 4,923 | (33.2) | 3,678 | (29.5) | <0.001 |
| Donor given insulin* | 16,495 | (60.4) | 8,558 | (57.7) | 7,937 | (63.7) | <0.001 |
| Recipient | | | | | | | |
| Recipient lung disease | | | | | | | <0.001 |
| Obstructive | 7,833 | (28.7) | 3,901 | (26.3) | 3,932 | (31.6) | |
| Pulmonary vascular | 1,041 | (3.8) | 638 | (4.3) | 403 | (3.2) | |
| Infectious | 2,621 | (9.6) | 1,575 | (10.6) | 1,046 | (8.4) | |
| Restrictive | 15,801 | (57.9) | 8,721 | (58.8) | 7,080 | (56.8) | |
| Recipient pre-transplant FEV1% | | | | | | | 0.064 |
| >80 | 1,849 | (6.8) | 1,043 | (7.0) | 806 | (6.5) | |
| 50-79 | 7,119 | (26.1) | 3,883 | (26.2) | 3,236 | (26.0) | |
| 30-49 | 7,655 | (28.0) | 4,200 | (28.3) | 3,455 | (27.7) | |
| <30 | 10,673 | (39.1) | 5,709 | (38.5) | 4,964 | (39.8) | |
| Recipient functional status | | | | | | | <0.001 |
| Independent | 1,685 | (6.2) | 722 | (4.9) | 963 | (7.7) | |
| Partial assist | 14,341 | (52.5) | 7,744 | (52.2) | 6,597 | (52.9) | |
| Total assist | 10,933 | (40.1) | 6,184 | (41.7) | 4,749 | (38.1) | |
| Unknown | 337 | (1.2) | 185 | (1.2) | 152 | (1.2) | |
| Recipient creatinine, normal | 25,935 | (95.0) | 14,178 | (95.6) | 11,757 | (94.4) | |
| Recipient history of diabetes | 5,318 | (19.5) | 2,851 | (19.2) | 2,467 | (19.8) | 0.23 |
| Recipient with previous malignancy | 2,360 | (8.6) | 1,268 | (8.5) | 1,092 | (8.8) | |
| Recipient requiring IV antibiotics\hat{ } | 2,785 | (10.2) | 1,533 | (10.3) | 1,252 | (10.0) | 0.44 |
| Recipient with prior lung surgery\hat{ } | 1,509 | (5.5) | 741 | (5.0) | 768 | (6.2) | <0.001 |
| Recipient received blood transfusion\hat{ } | 1,415 | (5.2) | 750 | (5.1) | 665 | (5.3) | 0.30 |
| Recipient condition at transplant | | | | | | | |
| ICU hospitalization | 2,984 | (10.9) | 1,737 | (11.7) | 1,247 | (10.0) | <0.001 |
| Non-ICU hospitalization | 2,547 | (9.3) | 1,419 | (9.6) | 1,128 | (9.1) | |
| Not hospitalized | 21,765 | (79.7) | 11,679 | (78.7) | 10,086 | (80.9) | |
| Recipient on ECMO at transplant | 1,134 | (4.2) | 742 | (5.0) | 392 | (3.1) | <0.001 |
| Recipient on any life support at | 2,686 | (9.8) | 1,531 | (10.3) | 1,155 | (9.3) | 0.004 |
| transplant | | | | | | | |
| Recipient ventilator support post- | | | | | | | <0.001 |
| transplant | | | | | | | |
| None | 936 | (3.4) | 384 | (2.6) | 552 | (4.4) | |
| ≤48 hours | 16,618 | (60.9) | 9,414 | (63.5) | 7,204 | (57.8) | |
| >48 hours & ≤5 days | 4,494 | (16.5) | 2,547 | (17.2) | 1,947 | (15.6) | |
| >5 days | 5,248 | (19.2) | 2,490 | (16.8) | 2,758 | (22.1) | |
| Recipient reintubated post-transplant | 4,832 | (17.7) | 2,201 | (14.8) | 2,631 | (21.1) | <0.001 |
| Recipient post-transplant airway | 383 | (1.4) | 147 | (1.0) | 236 | (1.9) | <0.001 |
| dehiscence | | | | | | | |
| Recipient post-transplant dialysis | 1,663 | (6.1) | 562 | (3.8) | 1,101 | (8.8) | <0.001 |
| Recipient post-transplant stroke | 587 | (2.2) | 228 | (1.5) | 359 | (2.9) | <0.001 |
| Length of stay, median (IQR) | 17 | (12-28) | 16 | (12-25) | 17 | (11-31) | <0.001 |
| Recipient survival time (days), median | 1,096 | (392-2,176) | 1,110 | (389-2,200) | 998 | (396-2,006) | <0.001 |
| (IQR) | | | | | | | |
| Allocation type | | | | | | | <0.001 |
| Local | 12,554 | (46.0) | 6,423 | (43.3) | 6,131 | (49.2) | |
| Regional | 5,808 | (21.3) | 3,480 | (23.5) | 2,328 | (18.7) | |
| National | 8,934 | (32.7) | 4,932 | (33.2) | 4,002 | (32.1) | |
| Distance donor to transplant center | 146 | (28-314) | 151 | (35-307) | 137 | (23-325) | 0.004 |
| (miles), median (IQR) | | | | | | | |
| Ischemic time (hours), median (IQR) | 5.15 | (4.12-6.25) | 5.28 | (4.27-6.38) | 4.98 | (3.97-6.08) | <0.001 |
| Transplant type | | | | | | | <0.001 |
| Single | 8,175 | (29.9) | 3,414 | (23.0) | 4,761 | (38.2) | |
| Double | 19,121 | (70.1) | 11,421 | (77.0) | 7,700 | (61.8) | |
| UNOS Region where transplanted | | | | | | | |
| 1 | 773 | (2.8) | 433 | (2.9) | 340 | (2.7) | <0.001 |
| 2 | 4,156 | (15.2) | 2,134 | (14.4) | 2,022 | (16.2) | |
| 3 | 2,822 | (10.3) | 1,505 | (10.1) | 1,317 | (10.6) | |
| 4 | 3,215 | (11.8) | 1,637 | (11.0) | 1,578 | (12.7) | |
| 5 | 4,376 | (16.0) | 2,450 | (16.5) | 1,926 | (15.5) | |
| 6 | 546 | (2.0) | 364 | (2.5) | 182 | (1.5) | |
| 7 | 2,216 | (8.1) | 1,189 | (8.0) | 1,027 | (8.2) | |
| 8 | 1,748 | (6.4) | 1,013 | (6.8) | 735 | (5.9) | |
| 9 | 1,180 | (4.3) | 687 | (4.6) | 493 | (4.0) | |
| 10 | 3,470 | (12.7) | 1,951 | (13.2) | 1,519 | (12.2) | |
| 11 | 2,794 | (10.2) | 1,472 | (9.9) | 1,322 | (10.6) | |

All displayed as n (%) unless otherwise specified.
CMV—cytomegalovirus.
FEV1—forced expiratory volume in one second.
ECMO—extracorporeal membrane oxygenation.
*Within 24 hours of procurement.
\hat{ }Between listing and transplant

TABLE 3
Survival tree subgroup definitions and estimated survivals

| | | Estimated survival, % | | |
|---|---|---|---|---|
| Sub- | | 1- | 5- | 10- |
| group | Subgroup description | year | year | year |
| 1 | Recipient age <64, LOS <50, Days of | 96 | 73 | 48 |
| | post-operative ventilator support <4, | | | |
| | Double lung transplantation, | | | |
| | No reintubation, Donor CMV negative | | | |
| 2 | Recipient age <64, LOS <50, Days of | 95 | 67 | 43 |
| | post-operative ventilator support <4, | | | |
| | Double lung transplantation, | | | |
| | No reintubation, Donor CMV positive | | | |
| 3 | Recipient age <64, LOS <50, Days | 87 | 60 | 36 |
| | of post-operative ventilator support <4, | | | |
| | Double lung transplantation, | | | |
| | Required reintubation | | | |
| 4 | Recipient age <64, LOS <50, Days of | 92 | 58 | 27 |
| | post-operative ventilator | | | |
| | support <4, Single lung | | | |
| | transplantation | | | |
| 5 | Recipient age <64, LOS <50, | 82 | 55 | 32 |
| | Days of post-operative | | | |
| | ventilator support ≥4 | | | |
| 6 | Recipient age <64, LOS ≥50 | 74 | 38 | 19 |
| 7 | Recipient age ≥64, LOS <53 | 89 | 52 | 20 |
| 8 | Recipient age ≥64, LOS ≥53 | 60 | 21 | 6 |

LOS—Length of stay (in days).
CMV—Cytomegalovirus.

Modelling: Sixty preoperative and immediate postoperative variables were evaluated with stepwise logistic regression, which returned 48 significant factors. One variable was removed due to a high degree of multicollinearity. Thus, 47 variables were included in all further analysis. The resulting 47-variable logistic regression model demonstrated acceptable accuracy (0.653; 95% confidence interval).

The survival tree analysis resulted in eight subgroups of patients, based on six variables: recipient age, LOS, duration of post-transplant ventilator support, transplant type (single vs. double lung), recipient reintubation post-transplant, and donor CMV positivity. Each subgroup has a distinct Kaplan-Meier survival curve (p<0.05 among all by log rank pairwise comparisons except for Subgroup 1 vs. 4 and Subgroup 1 vs. 5). The one, five, and 10-year estimated survival probabilities were predicted for each respective subgroup (Table 3). Among the eight subgroups, the recipients who were younger than 64 years old, had length of stay less than 50 days, less than four days of post-operative ventilator support, underwent double lung transplantation, required no reintubation, and had a CMV-negative donor (Subgroup 1) had the best predicted survival outcomes with 96% survival at one year, 73% at five years, and 48% at 10 years. Subgroup 2 was the same as Subgroup 1, except they received organs from CMV-positive donors, which caused only a 1% decrease in one-year survival, but a 6% decrease in five-year survival, and a 5% decrease in 10-year survival. For patients age 64 or older, only LOS significantly impacted their survival, with Subgroup 7 (age 64 or older, and LOS less than 53 days) having a one-year survival of 89%, while Subgroup 8 (age 64 or older, and LOS 53 days or more) had a 60% one-year survival. Subgroup 8 had very low five- and 10-year survivals at 21% and 6%, respectively.

Discussion: Despite advancement in surgical techniques and evolution of medicine, the survival rates following lung transplant remain low. This issue underscores the need of accurate and personalized survival prediction, a critical resource to support clinical decision making and ultimately enhance post-transplant survival. While this logistic regression found several donor and pre- and post-operative recipient factors associated with recipient mortality after lung transplant, the ML survival tree methodology preferentially selected factors of recipient age, LOS, duration of post-transplant ventilator support, transplant type (single vs. double lung), recipient reintubation post-transplant, and donor CMV positivity. The significance of the present study lies not only in the survival tree algorithm's ability to identify pivotal prognostic factors, but also the capacity to unveil the hierarchical importance among these clinical factors.

Survival tree algorithms construct a binary classification system by leveraging the variable with the highest discriminatory power in predicting outcomes. The primary advantage associated with survival tree modeling lies in its simplicity, as it permits the direct identification of interactions between variables through the model, and the ability to convey probabilities within the decision tree. As it is an easily interpretable model, it provides significant clinical utility. This straightforward structural design not only enhances comprehension for both surgeons and patients but also distinguishes itself from more complex statistical methodologies. This simplicity enhances not only the pre-operative conversation with the patient but also, as the patient experiences post-operative complications, the surgical team is better able to guide and inform patients about how these changes now affect their expected long-term survival.

The survival tree identified two preoperative factors with significant implications for prognosis: recipient age and donor CMV positivity. Notably, recipient age emerged as the most pivotal prognostic factor. While advanced age does not serve as an absolute contraindication for lung transplantation, previous research indicates that older individuals, particularly those aged 70 years and above, experience worse long-term prognoses. Additionally, donor CMV status was recognized as a crucial determinant of survival, which is in line with existing literature that reports CMV infection as a substantial risk factor for lung transplant recipients. Primary prevention with antiviral agents after transplantation reduces the rate of CMV infection, and the resulting risks of CMV pneumonitis and mortality. Consequently, these findings have prompted the adoption of CMV prophylaxis regimens following transplantation.

In the realm of intra- and post-operative factors, the survival tree model identified several key variables, including transplant type (single vs. double lung), duration of hospitalization from transplant to discharge, length of post-transplant ventilator support, and recipient reintubation post-transplant. However, double lung transplantation exhibited a more favorable prognosis in comparison to single lung transplant, aligning with previous long-term survival data. The use of bilateral lungs can ameliorate complications associated with the contralateral native lung in single lung transplantation, such as the risk of infection due to structurally damaged lung, susceptibility to malignancies in the native lung, and the potential compromise of the allograft due to native lung hyperinflation in COPD patients.

Additionally, prolonged LOS was associated with worse survival probabilities. Other factors such as longer post-transplant ventilator support and need for reintubation after transplant were also associated with worse survival probabilities in this study. These significant post-operative variables shed light on the complex cadre of factors that influence patient survival after lung transplantation, and highlight the limitations of considering only pre-operative factors when counseling patients.

The inclusion of patients who had received a prior lung transplant can also be mentioned; they made up 7.5% of our cohort. These patients were included as a priori hypothesis that prior lung transplant would have an influence on survival was obtained. The final logistic regression model did indeed confirm this, with patients who had a prior lung transplant having 1.65 times higher odds of mortality than patients who had not had prior lung transplant (Odds Ratio 1.65 [95% confidence interval 1.41-1.93]). While there appears to be a significant association between prior lung transplant and mortality, the survival tree model did not include this variable. This does not mean it is not influential on patient survival, but rather highlights the goal of survival tree algorithms to parse patients into subgroups based on the variables that most effect survival.

As discussed, the survival tree included two pre-operative and four post-operative variables. This is highly important for clinicians and patients as it indicates survival following lung transplantation cannot be adequately predicted by pre-operative factors alone. It therefore must be explained to patients that their survival, while influenced by their age at transplant, their donor's CMV status, and receipt of single or double lung transplant, will also be contingent upon their post-operative course—namely, their LOS, duration of ventilator support, and need for reintubation. While this may not be satisfying for the pre-operative patient, it is important to convey limitations to pre-operative survival predictions and explain that some of their survival estimation will be based on their initial post-operative course.

### Example #2

Methods: The United Network Organ Sharing (UNOS) Standard Transplant Analysis and Research (STAR) database was queried for all orthotopic liver transplants in recipients ≥18 years old between 2000-2021. The UNOS STAR files contain national patient-level information regarding transplant recipient, deceased and living donors, and waitlist candidates reported to the Organ Procurement and Transplant Network (OPTN) from all transplant centers since October 1987. All variables available with less than 10% missing data were included. Data collection year range was chosen to encompass the modern era of calcineurin inhibitors. Continuous laboratory values (i.e., total bilirubin, serum albumin) were converted to categorical variables for modeling purposes. Donor and recipient factors were evaluated with multivariable logistic regression with a combination of forward selection and backward elimination. Instances of graft survival <7 days were excluded. Following significant variable selection, a total of 43 factors were used in survival tree and prediction modeling. Data were split into 70% and 30% training and testing sets, respectively, and further validated with ten-fold cross validation.

Survival tree modeling was performed by implementing the SurvCART algorithm. This technique utilizes the conditional inference framework that selects the splitting variable by parameter instability test and subsequently finds the optimal split based on a maximally chosen statistic (e.g., maximum log-rank test statistic). Further, the algorithm is able to consider the heterogeneity of survival times and extend this to the censoring distribution in cases where marker dependent censoring ignorance of the censoring distribution may lead to inconsistent trees. The algorithm thereby elicits the information on the association of covariates and their interaction on the survival outcome in a data driven manner. The survival tree was fitted using the ‘SurvCART( )’ function, and Kaplan-Meier (KM) estimate of survival probabilities for the extracted subgroups were obtained using the “predict( )’ function from the ‘LongCART’ package in R (R Core Team, Vienna, Austria). In addition to producing yearly survival probabilities, pair-wise log rank test was used to determine statistical significance among the survival curves of various subgroups. Pair-wise rank test was conducted from the R package ‘survminer’ using the ‘pairwise_survdiff’ function.

The Area Under the Curve Receiver Operator Characteristic (AUC-ROC) curves were generated for model performance evaluation and Kaplan-Meier plots produced for each respective subgroup. Survival tree pruning and model selection was performed based on Akaike information criterion (AIC) and log-likelihood values. Data preparation, merging, and cleaning were performed using Stata software version 16 (StataCorp, College Station, TX). Modeling and survival probability calculations, including implementation of the SurvCART algorithm, was performed in R Studio Statistical Analysis software. Descriptive continuous variables are presented as median (standard deviation) and categorical variables as n (%). This study was deemed exempt from the University of South Florida institutional review board review given the de-identified retrospective database nature of this analysis.

Results—Study Population: A total of 122,134 liver transplant patients between 2000 and 2021 were included for analysis. Overall, recipients were a median age of 54 years old, majority male (66.67%), and spent a median of 247 total days on the transplant wait list. The majority of recipients had private health insurance (55.9%), had completed high school or educational equivalent, and were non-diabetic. Liver transplant donors were younger (median age 43 years old), majority non-Hispanic males. Deceased patients spent an average of 10 days longer on the transplant wait list (257 vs 247 days, p<0.001), were more likely to have undergone previous liver transplant (8.3% vs 4.8%, p<0.001), and more likely to have Hepatitis C infection at the time of transplant (41.6% vs 30.7%, p<0.001).

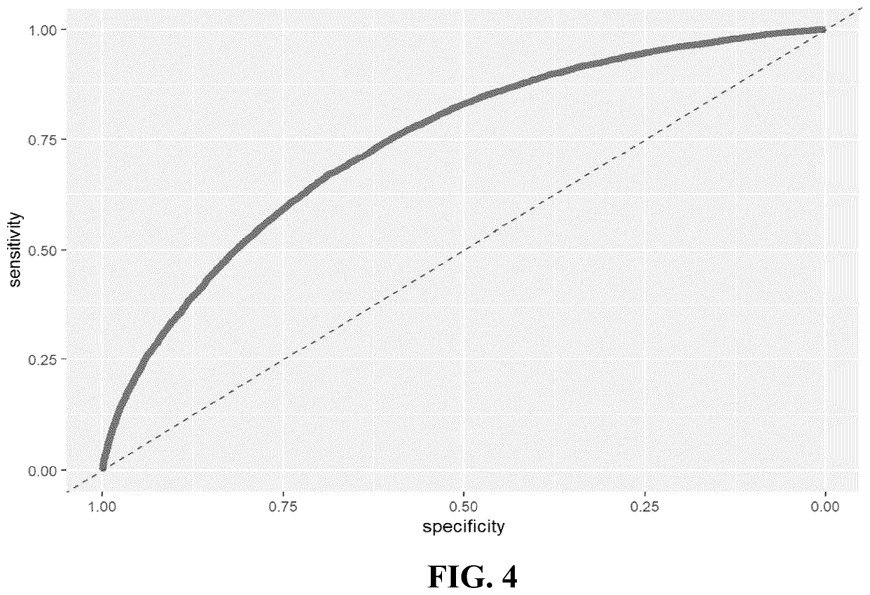

Multivariable Logistic Regression: Multivariable logistic regression was performed with 134 donor, recipient, and hospital-level variables. Modeling performance was satisfactory (accuracy=0.728; 95% confidence interval [CI] 0.724-0.733; AUC=0.742, F1=0.822, FIG. 4). A total of 43 significant donor, recipient, and donor-center factors remained after stepwise selection. The top ten most significant factors by logit modeling included the recipient's functional status at the time of transplant listing, the presence of malignancy in the recipient, recipient diabetes, recipient level of education achieved, and blood type AB.

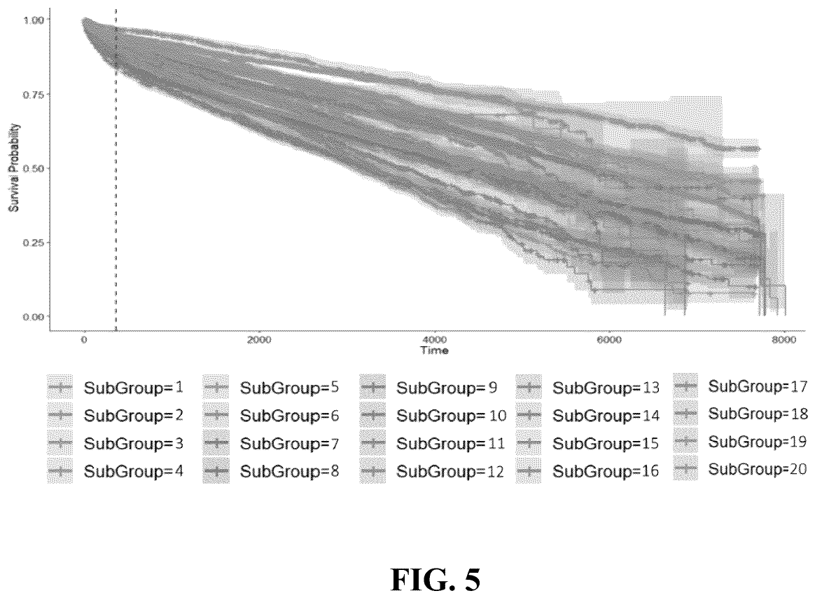

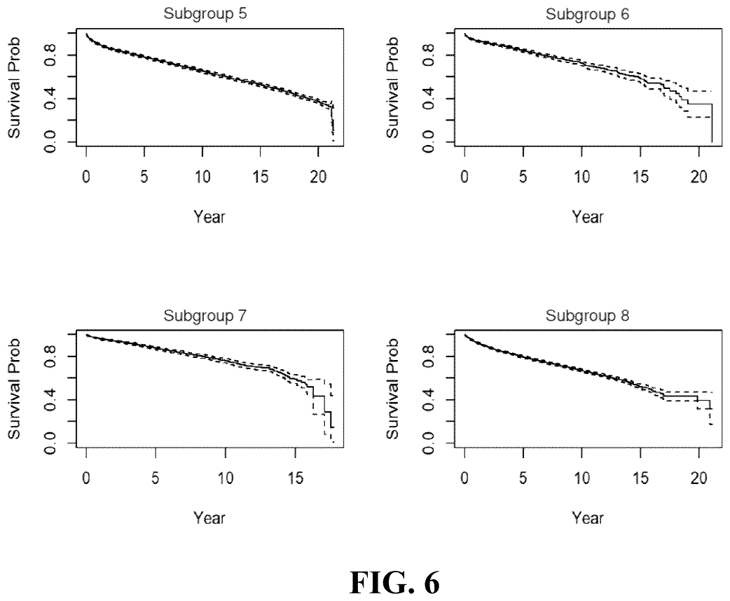

Survival Tree and Mortality Prediction Modeling: Survival tree modeling returned eight significant recipient survival factors: recipient age, donor age, recipient primary payment, recipient Hepatitis C status, recipient diabetes, recipient functional status at registration and at transplantation, and deceased donor pulmonary function. Twenty subgroups consisting of combinations of these factors were identified with distinct subgroup Kaplan-Meier survival curves (p<0.001 among all by log rank pair wire comparison, FIG. 5). Kaplan-Meier survival curves by subgroup were also generated with 95% confidence intervals (FIG. 6). Five (overall range 68-88%) and ten-year (overall range 43-76%) survival probabilities were predicted for each respective subgroup.

Discussion: Various liver transplant recipient, donor, donor-center, and other factors influence long-term patient survival. The complex interplay of these variables on patient outcome is often difficult to determine and may result in a miscalculation of survival probability using traditional linear statistical techniques. Here, an innovatively applied survival tree and prediction algorithm to the UNOS liver transplant data is reported to generate an easily interpretable survival model with one-, five-, and ten-year estimated survival probabilities.

Accurately predicting long term patient survival following liver transplantation may assist in transplant decision making and allow for more coherent patient and family counseling discussions. While current scoring systems have shown proficiency for predicting waitlist mortality or prognosis of cirrhosis, few tools are currently available to reliably predict long-term mortality following transplant or are able to elucidate the degree to which covariate interactions impact mortality. Though SOFT and BAR scores have improved post-transplant mortality predictive accuracy compared to MELD, one and five-year predictive performance was unsatisfactory in validation studies. Additionally, these methods use a small number of clinical variables and do not consider social or physical factors, including educational achievement, insurance status, or a patient's functional status. These results highlight the need for a more accurate post-liver transplant mortality prediction model. The ReSOLT risk calculator is able to provide long term post-transplant mortality prediction and details on covariate interaction using the most significant recipient and donor factors by survival tree modeling. By providing mortality prediction using a small number of variables, clinicians can utilize ReSOLT in the context of other clinical parameters and risk calculators to offer a comprehensive interpretation of a patient's clinical course pre- and post-transplant. Further, identification of salient patient factors (i.e., patient functional status) may allow for patient optimization prior to transplant, resulting in decreased long-term mortality risk. While other risk scoring models utilize laboratory values as a predictor of patient outcome, ReSOLT modeling returned variables that perhaps have a larger impact on patient survival in the long-term. Though hyperbilirubinemia or hyponatremia may have profound impact in short-term waitlist survival, these factors are often corrected in the post-transplant patient and may not contribute to overall long-term mortality. These findings highlight the importance of donor and recipient age, functional capacity, and health insurance status as important factors for long-term survival following transplantation.

Determining the effect of covariate interactions on survival distribution traditionally requires pre-specification of the functional form of the covariates, including their interactions. The SurvCART methodology utilizes the “conditional inference” framework to select the splitting variable via parameter instability test and subsequently finds the optimal split. The result is an inimitable and flexible approach to determining covariate interaction. In this analysis, application of this technique resulted in a unique understanding of covariate effect on survival probability. As the survival tree is constructed, branch points provide information on the effect of variable changes on long term mortality. For example, liver transplant subgroups 17 and 18 differ by recipient Hepatitis C status at the time of transplant, resulting in an estimated mortality probability difference of 12% and 13% at five and ten years, respectively. Determination of the degree to which variables impact survival could allow for targeted optimization to mitigate predicted long-term mortality (i.e., improving functional status, recipient treatment of Hepatitis C prior to transplant). Currently, organ allocation is stratified by waitlist mortality probability without consideration for long-term patient outcome. While one-year survival following liver transplant is excellent in the modern era, long-term patient specific survival remains nuanced and is not currently included in organ allocation systems. Utilization of techniques that include long-term survival may assist in transplant decision making or provide opportunity for intervention on modifiable factors resulting in increased post-transplant survival.

### Example #3

Methods: The United Network Organ Sharing (UNOS) Standard Transplant Analysis and Research database was queried for all orthotopic liver transplants in recipients ≥18 years old between 2000 and 2021. The UNOS Standard Transplant Analysis and Research files contain national patient-level information regarding transplant recipient, deceased and living donors, and waitlist candidates reported to the Organ Procurement and Transplant Network from all transplant centers since October 1987. All variables available with <10% missing data were included. The data collection year range was chosen to encompass the modern era of calcineurin inhibitors. Continuous laboratory values (ie total bilirubin, serum albumin) were converted to categorical variables for modeling purposes. Donor and recipient factors were evaluated with multivariable logistic regression with a combination of forward selection and backward elimination. Instances of graft survival of <7 days were excluded. Following significant variable selection, a total of 43 factors were used in survival tree and prediction modeling. The data were split into 70% and 30% training and testing sets, respectively, and further validated with 10-fold cross-validation.

Survival tree modeling was performed by implementing the SurvCART algorithm. This technique uses the conditional inference framework that selects the splitting variable by parameter instability test and subsequently finds the optimal split based on a maximally chosen statistic (eg maximum log-rank test statistic). Further, the algorithm is able to consider the heterogeneity of survival times and extend this to the censoring distribution in cases where marker-dependent censoring ignorance of the censoring distribution may lead to inconsistent trees. The algorithm thereby elicits the information on the association of covariates and their interaction on the survival outcome in a data-driven manner. The survival tree was fitted using the “SurvCART( )” function, and Kaplan-Meier estimates of survival probabilities for the extracted subgroups were obtained using the “predict( )” function from the “LongCART” package in R (R Core Team, Vienna, Austria). In addition to producing yearly survival probabilities, a pair-wise log rank test was used to determine statistical significance among the survival curves of various subgroups. The pair-wise rank test was conducted from the R package “survminer” using the “pairwise_survdiff” function.

AUC receiver operator characteristic curves were generated for model performance evaluation, and Kaplan-Meier plots were produced for each respective subgroup. Survival tree pruning and model selection was performed based on Akaike information criterion and log-likelihood values. Data preparation, merging, and cleaning were performed using Stata software version 16 (StataCorp, College Station, TX). Modeling and survival probability calculations, including implementation of the SurvCART algorithm, was performed in R Studio Statistical Analysis software. Descriptive continuous variables are presented as median (standard deviation) and categorical variables as n (%). This study was deemed exempt from the University of South Florida institutional review board review given the deidentified retrospective database nature of this analysis.

Results—Study Population: A total of 122,134 liver transplant patients between 2000 and 2021 were included for analysis. Overall, recipients were a median age of 54 years old, the majority were male (66.67%), and they spent a median of 247 total days on the transplant wait list (Table 4). The majority of recipients had private health insurance (55.9%), had completed high school or the educational equivalent, and were nondiabetic. Liver transplant donors were younger (median age 43 years), majority non-Hispanic males. Deceased patients spent an average of 10 days longer on the transplant wait list (257 vs 247 days; p<0.001), were more likely to have undergone previous liver transplant (8.3% vs 4.8%; p<0.001), and were more likely to have hepatitis C infection at the time of transplant (41.6% vs 30.7%; p<0.001; Table 5).

TABLE 4
Donor and Recipient Baseline Liver Transplant Patient Demographics

| | Alive | | Deceased | | Total | | |
|---|---|---|---|---|---|---|---|
| Variable | (n = 84,280) | | (n = 37,854) | | (n = 122,134) | | p Value |
| Recipient age, y, | 54 | (11) | 55 | (10) | 54 | (11) | <0.001 |
| median (SD) | | | | | | | |
| Donor age, y, | 42 | (15) | 44 | (15) | 43 | (15) | <0.001 |
| median (SD) | | | | | | | |
| Total days on | 244 | (465) | 254 | (456) | 247 | (462) | <0.001 |
| waiting list | | | | | | | |
| (including inactive | | | | | | | |
| time), median (SD) | | | | | | | |
| Donor sex, n (%) | | | | | | | 0.002 |
| Female | 33,577 | (39.84) | 15,435 | (40.78) | 49,012 | (40.13) | |
| Male | 50,703 | (60.16) | 22,419 | (59.222) | 73,122 | (59.87) | |
| Recipient sex, n (%) | | | | | | | <0.001 |
| Female | 28,968 | (34,37) | 11,738 | (31.01) | 40,706 | (33.33) | |
| Male | 55,312 | (65.63) | 26,116 | (68.99) | 81,428 | (66.65) | |
| Ethnicity, n (%) | | | | | | | <0.001 |
| Non-Hispanic | 71,764 | (85.1) | 33,317 | (88.0) | 105,081 | (86.0) | |
| Hispanic | 12,516 | (14.9) | 4,537 | (12.0) | 17,053 | (14.0) | |
| Donor BMI at | | | | | | | <0.001 |
| donation, n (%) | | | | | | | |
| Normal | 29,458 | (35.0) | 14,024 | (37.0) | 43,482 | (35.6) | |
| Underweight | 2,620 | (3.1) | 1,319 | (3.5) | 3,939 | (3.2) | |
| Overweight | 27,989 | (33.2) | 12,832 | (33.9) | 40,821 | (33.4) | |
| Obese | 24,213 | (28.7) | 9,679 | (25.6) | 33,892 | (27.7) | |
| Recipient BMI at | | | | | | | <0.001 |
| listing, n (%) | | | | | | | |
| Normal | 20,867 | (24.8) | 9,783 | (25.8) | 30,650 | (25.1) | |
| Underweight | 999 | (1.2) | 526 | (1.4) | 1,525 | (1.2) | |
| Overweight | 29,832 | (35.4) | 13,835 | (36.5) | 43.667 | (35.8) | |
| Obese | 32,582 | (38.7) | 13,710 | (36.2) | 46,292 | (37.9) | |
| Previous liver | | | | | | | <0.001 |
| transplant, n (%) | | | | | | | |
| No | 80,242 | (95.2) | 34,723 | (91.7) | 114,965 | (94.1) | |
| Yes | 4,038 | (4.8) | 3,131 | (8.3) | 7,169 | (5.9) | |
| Recipient highest | | | | | | | |
| education level, n | | | | | | | |
| (%) | | | | | | | |
| None | 279 | (0.3) | 101 | (0.3) | 380 | (0.3) | |
| Grade school | 3,777 | (4.5) | 1,595 | (4.2) | 5,372 | (4.4) | |
| High School or GED | 32,351 | (38.4) | 15,011 | (39.7) | 47,362 | (38.8) | |
| Attended | 19,464 | (23.1) | 7,486 | (19.8) | 26,950 | (22.1) | |
| college/technical | | | | | | | |
| school | | | | | | | |
| Associate/bachelor's | 14,651 | (17.4) | 4,701 | (12.4) | 19,352 | (15.8) | |
| degree | | | | | | | |
| Post-college | 5,795 | (6.9) | 1,981 | (5.2) | 7,776 | (6.4) | |
| graduate degree | | | | | | | |
| Others | 23 | (0.0 | 22 | (0.1) | 45 | (0.0) | |
| Unknown | 7,940 | (9.4) | 6,957 | (18.4) | 14,897 | (12.2) | |
| Previous transplant | | | | | | | <0.001 |
| of any organ type in | | | | | | | |
| OPTN database, n | | | | | | | |
| (%) | | | | | | | |
| No | 79,897 | (94.8) | 34,460 | (91.0) | 114,357 | (93.6) | |
| Yes | 4,383 | (5.2) | 3,394 | (9.0) | 7,777 | (6.4) | |
| Graft status, n (%) | | | | | | | <0.001 |
| Not failed | 79,805 | (94.7) | 1,588 | (4.2) | 81,393 | (66.6) | |
| Failed | 4,475 | (5.3) | 36,266 | (95.8) | 40,741 | (33.4) | |
| Recipient primary | | | | | | | <0.001 |
| source of payment, n | | | | | | | |
| (%) | | | | | | | |
| Private insurance | 47,823 | (56.7) | 20,473 | (54.1) | 68,296 | (55.9) | |
| Public insurance - | 12,362 | (14.7) | 5,445 | (14.1) | 17,697 | (14.5) | |
| Medicaid | | | | | | | |
| Public insurance - | 11,912 | (14.1) | 5,210 | (13.8) | 17,122 | (14.0) | |
| Medicare Fee for | | | | | | | |
| Service | | | | | | | |
| Public Insurance - | 7,424 | (8.8) | 3,227 | (8.5) | 10,651 | (8.7) | |
| Medicare & Choice | | | | | | | |
| Public Insurance - | 9 | (0.0) | 3 | (0.0) | 12 | (0.0) | |
| Children's Health | | | | | | | |
| Insurance Program | | | | | | | |
| Public Insurance - | 1,747 | (2.1) | 930 | (2.5) | 2,677 | (2.2) | |
| Department of VA | | | | | | | |
| Public Insurance - | 957 | (1.1) | 267 | (0.7) | 1,224 | (1.0) | |
| Other government | | | | | | | |
| Self | 455 | (0.5) | 154 | (0.4) | 609 | (0.5) | |
| Free Care | 138 | (0.2) | 67 | (0.2) | 205 | (0.2) | |
| Donation | 16 | (0.0) | 13 | (0.0) | 29 | (0.0) | |
| Foreign government | 330 | (0.4) | 63 | (0.2) | 393 | (0.3) | |
| specify | | | | | | | |
| Public insurance - | 939 | (1.1) | 1,899 | (5.0) | 2,838 | (2.3) | |
| Medicare | | | | | | | |
| (unspecified) | | | | | | | |
| US/State | 168 | (0.2) | 213 | (0.6) | 381 | (0.3) | |
| government agency | | | | | | | |

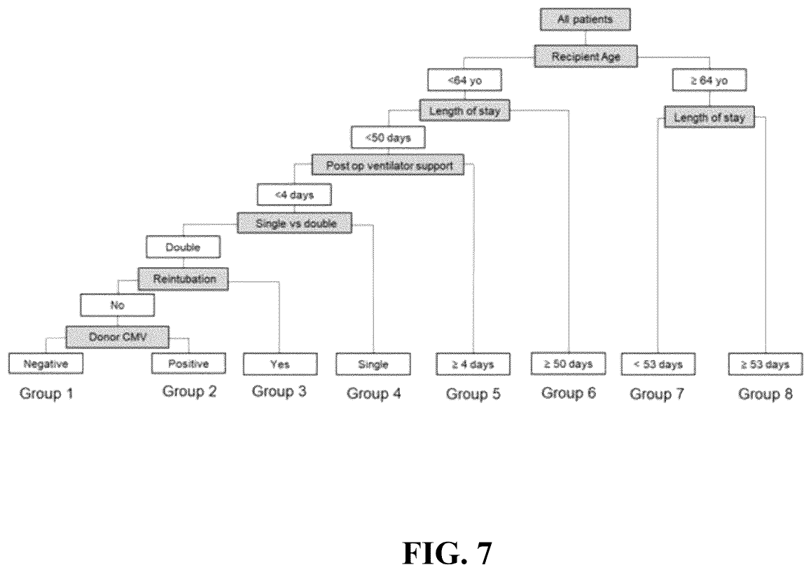

Multivariable Logistic Regression: Multivariable logistic regression was performed with 134 donor, recipient, and hospital-level variables. Modeling performance was satisfactory (accuracy=0.728; 95% CI=0.724 to 0.733; AUC=0.742, F1=0.822; FIG. 7). A total of 43 significant donor, recipient, and donor-center factors remained after stepwise selection. The top 10 most significant factors by logit modeling included the recipient's functional status at the time of transplant listing, the presence of malignancy in the recipient, recipient diabetes, recipient level of education achieved, and blood type AB.

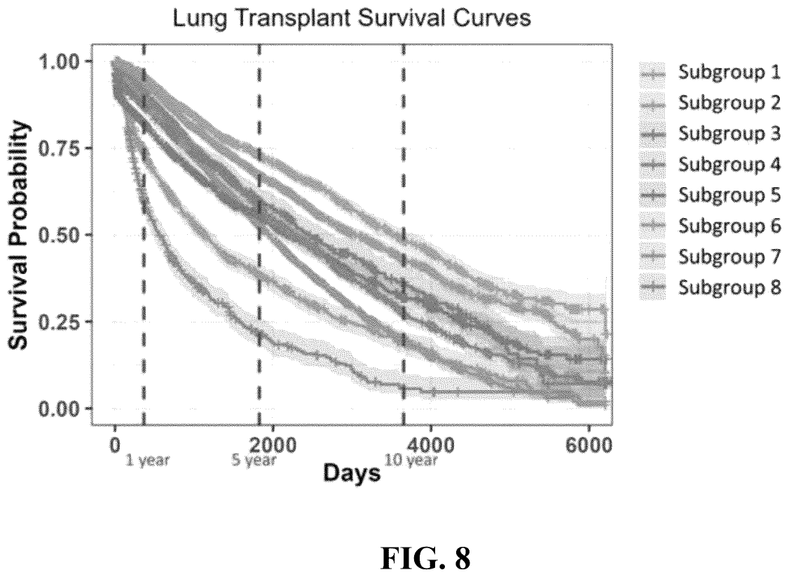

Survival Tree and Mortality Prediction Modelling: Survival tree modeling returned 8 significant survival factors: recipient age, donor age, recipient primary payment, recipient hepatitis C status, recipient diabetes, recipient functional status at registration and at transplantation, and deceased donor pulmonary function. Twenty subgroups consisting of combinations of these factors were identified with distinct subgroup Kaplan-Meier survival curves (p<0.001 among all by log rank pair wire comparison; FIG. 8). Kaplan-Meier survival curves by subgroup were also generated with 95% confidence intervals. The 5-year (overall range 68 to 88%) and 10-year (overall range 43 to 76%) survival probabilities were predicted for each respective subgroup (Table 6).

TABLE 5
Recipient and Donor Liver Transplant Clinical Variables

| | Alive | | Deceased | | Total | | |
|---|---|---|---|---|---|---|---|
| Variable | (n = 84,280) | | (n = 37,854) | | (n = 122,134) | | p Value |
| Deceased donor | | | | | | | <0.001 |
| pulmonary | | | | | | | |
| infection | | | | | | | |
| No | 40,249 | (47.8) | 24,563 | (64.9) | 64,812 | (53.1) | |
| Yes | 44,031 | (52.2) | 13,291 | (35.1) | 57,322 | (46.9) | |
| Transplant | | | | | | | <0.001 |
| recipient | | | | | | | |
| diabetes history | | | | | | | |
| No | 63,074 | (74.8) | 26,034 | (68.8) | 89,108 | (73.0) | |
| 0-5 y | 1,022 | (1.2) | 820 | (2.2) | 1,842 | (1.5) | |
| 6-10 y | 16,795 | (19.9) | 6,754 | (17.8) | 23,549 | (19.3) | |
| >10 y | 401 | (0.5) | 110 | (0.3) | 511 | (0.4) | |
| Duration | 2,204 | (2.6) | 3,418 | (9.0) | 5,622 | (4.6) | |
| unknown | | | | | | | |
| Unknown status | 784 | (0.9) | 718 | (1.9) | 1,502 | (1.2) | |
| Donor diabetes | | | | | | | <0.001 |
| history | | | | | | | |
| No | 74,717 | (88,7) | 33,725 | (89.1) | 108,442 | (88.8) | |
| 0-5 y | 3,068 | (3.6) | 1,412 | (3.7) | 4,480 | (3.7) | |
| 6-10 y | 1,649 | (2.0) | 699 | (1.8) | 2,348 | (1.9) | |
| >10 y | 3,309 | (3.9) | 1,402 | (3.7) | 4,711 | (3.9) | |
| Duration | 958 | (1.1) | 433 | (1.1) | 1,391 | (1.1) | |
| unknown | | | | | | | |
| Unknown | 579 | (0.7) | 183 | (0.5) | 762 | (0.6) | |
| Recipient on life | | | | | | | |
| support - | | | | | | | |
| ventilator at | | | | | | | |
| registration | | | | | | | |
| No | 81,755 | (97.0) | 36,421 | (96.2) | 118,176 | (96.8) | |
| Yes | 2,525 | (3.0) | 1,433 | (3.8) | 3,958 | (3.2) | |
| Recipient | | | | | | | <0.001 |
| hepatitis C status | | | | | | | |
| Not detected | 54,875 | (65.1) | 19,207 | (50.7) | 74,082 | (60.7) | |
| Present | 25,909 | (30.7) | 15,747 | (41.6) | 41,656 | (34.1) | |
| Unknown | 3,496 | (4.1) | 2,900 | (7.7) | 6,396 | (5.2) | |
| Donor history of | | | | | | | <0.001 |
| cocaine use | | | | | | | |
| No | 67,444 | (80.0) | 31,831 | (84.1) | 99,275 | (81.3) | |
| Unknown | 15,302 | (18.2) | 5,342 | (14.1) | 20,644 | (16.9) | |
| Yes | 1,534 | (1.8) | 681 | (1.8) | 2,215 | (1.8) | |
| Donor history of | | | | | | | <0.001 |
| hypertension | | | | | | | |
| No | 56,013 | (66.5) | 24,659 | (65.1) | 80,672 | (66.1) | |
| Unknown | 27,578 | (32.7) | 12,917 | (34.1) | 40,495 | (33.2) | |
| Yes | 689 | (0.8) | 278 | (0.7) | 967 | (0.8) | |
| Recipient any | | | | | | | <0.001 |
| previous | | | | | | | |
| malignancy | | | | | | | |
| No | 69,763 | (82.8) | 31,449 | (83.1) | 101,212 | (82.9) | |
| Unknown | 12,890 | (15.3) | 5,002 | (13.2) | 17,892 | (14.6) | |
| Yes | 1,627 | (1.9) | 1,403 | (3.7) | 3,030 | (2.5) | |
| Recipient TIPS | | | | | | | <0.001 |
| at registration | | | | | | | |
| No | 75,465 | (89.5) | 32,309 | (85.4) | 107,774 | (88.2) | |
| Unknown | 5,608 | (6.7) | 2,811 | (7.4) | 8,419 | (6.9) | |
| Yes | 3,207 | (3.8) | 2,734 | (7.2) | 5,941 | (4.9) | |
| Recipient | | | | | | | |
| spontaneous | | | | | | | |
| bacterial | | | | | | | |
| peritonitis at | | | | | | | |
| registration | | | | | | | |
| No | 76,074 | (90.3) | 33,854 | (89.4) | 109,928 | (90.0) | |
| Unknown | 6,660 | (7.9) | 2,596 | (6.9) | 9,256 | (7.6) | |
| Yes | 1,546 | (1.8) | 1,404 | (3.7) | 2,950 | (2.4) | |
| Donor given | | | | | | | <0.001 |
| synthetic | | | | | | | |
| antidiuretic | | | | | | | |
| hormone | | | | | | | |
| (ddAVP) | | | | | | | |
| No | 69,664 | (82.7) | 28,417 | (75.1) | 98,081 | (80.3) | |
| Unknown | 14,500 | (17.2) | 9,271 | (24.5) | 23,771 | (19.5) | |
| Yes | 116 | (0.1) | 166 | (0.4) | 282 | (0.2) | |
| Recipient | | | | | | | <0.001 |
| dialysis prior | | | | | | | |
| week to | | | | | | | |
| transplant | | | | | | | |
| No | 72,562 | (86.1) | 32,354 | (85.5) | 104,916 | (85.9) | |
| Unknown | 11,533 | (13.7) | 5,181 | (13.7) | 16,714 | (13.7) | |
| Yes | 185 | (0.2) | 319 | (0.8) | 504 | (0.4) | |
| Was transplant | | | | | | | 0.070 |
| exception ever | | | | | | | |
| submitted for | | | | | | | |
| patient | | | | | | | |
| No | 57,575 | (68.3) | 26,057 | (68.8) | 83,632 | (68.5) | |
| Yes | 26,705 | (31.7) | 11,797 | (31.2) | 38,502 | (31.5) | |
| Cadaver donor | | | | | | | <0.001 |
| received | | | | | | | |
| prerecovery | | | | | | | |
| medication | | | | | | | |
| No | 10,509 | (12.5) | 10,447 | (27.6) | 20,956 | (17.2) | |
| Yes | 73,771 | (87.5) | 27,407 | (72.4) | 101,178 | (82.8) | |
| Donor | | | | | | | |
| pulmonary | | | | | | | |
| infection | | | | | | | |
| No | 40,249 | (47.8) | 24,563 | (64.9) | 64,812 | (53.1) | |
| Yes | 44,031 | (52.2) | 13,291 | (35.1) | 57,322 | (46.9) | |
| Donor terminal | | | | | | | <0.001 |
| blood urea | | | | | | | |
| nitrogen | | | | | | | |
| Normal | 48,177 | (57.2) | 23,751 | (62.7) | 71,928 | (58.9) | |
| Low | 4,785 | (5.7) | 3,184 | (8.4) | 7,969 | (6.5) | |
| High | 31,318 | (37.2) | 10,919 | (28.8) | 42,237 | (34.6) | |
| Recipient serum | | | | | | | <0.001 |
| creatinine at time | | | | | | | |
| of transplant | | | | | | | |
| Normal | 48,142 | (57.1) | 20,069 | (53.0) | 68,211 | (55.8) | |
| Low | 1,453 | (1.7) | 482 | (1.3) | 1,935 | (1.6) | |
| High | 34,685 | (41.2) | 17,303 | (45.7) | 51,988 | (42.6) | |
| Recipient total | | | | | | | <0.001 |
| bilirubin at | | | | | | | |
| transplant | | | | | | | |
| Normal | 13,717 | (16.3) | 6,711 | (17.7) | 20,428 | (16.7) | |
| High | 70,563 | (83.7) | 31,143 | (82.3) | 101,706 | (83.3) | |
| Recipient serum | | | | | | | <0.001 |
| albumin at | | | | | | | |
| transplant | | | | | | | |
| Normal | 28,832 | (34.2) | 10,568 | (27.9) | 39,400 | (32.3) | |
| Low | 55,263 | (65.6) | 27,199 | (71.9) | 82,462 | (67.5) | |
| High | 185 | (0.2) | 87 | (0.2) | 272 | (0.2) | |

Discussion: Various liver transplant recipient, donor, donor-center, and other factors influence long-term patient survival. The complex interplay of these variables on patient outcome is often difficult to determine and may result in a miscalculation of survival probability using traditional linear statistical techniques. Here, we report an innovatively applied survival tree and prediction algorithm to the UNOS liver transplant data to generate an easily interpretable survival model with 1-, 5-, and 10-year estimated survival probabilities.

Accurately predicting long-term patient survival following liver transplantation may assist in transplant decision-making and allow for more coherent patient and family counseling discussions. While current scoring systems have shown proficiency for predicting waitlist mortality or prognosis of cirrhosis, few tools are currently available to reliably predict long-term mortality following transplant or are able to elucidate the degree to which covariate interactions affect mortality. Although SOFT and BAR scores have improved post-transplant mortality predictive accuracy compared to the MELD, 1- and 5-year predictive performance was unsatisfactory in validation studies. Additionally, these methods use a small number of clinical variables and do not consider social or physical factors, including educational achievement, insurance status, or a patient's functional status. These results highlight the need for a more accurate post liver transplant mortality prediction model. The ReSOLT risk calculator is able to provide long-term post-transplant mortality prediction and details on covariate interaction using the most significant recipient and donor factors by survival tree modeling. By providing mortality prediction using a small number of variables, clinicians can use ReSOLT in the context of other clinical parameters and risk calculators to offer a comprehensive interpretation of patient's clinical course pre- and post-transplant. Further, identification of salient patient factors (i.e., patient functional status) may allow for patient optimization prior to transplant, resulting in decreased long-term mortality risk. While other risk scoring models use laboratory values as a predictor of patient outcome, ReSOLT modeling returned variables that perhaps have a larger impact on patient survival in the long term. Although hyperbilirubinemia or hyponatremia may have profound impacts on short-term waitlist survival, these factors are often corrected in the post-transplant patient and may not contribute to overall long-term mortality. These findings highlight the importance of donor and recipient age, functional capacity, and health insurance status as important factors for long-term survival following transplantation.

TABLE 6
Recipient Subgroups Provided by Survival Tree Analysis
with Predicted 5- and 10-Year Mortality Estimation

| | | 5-year | 10-year |
|---|---|---|---|
| | | survival | survival |
| Subgroup | Subgroup Description | estimate, % | estimate, % |
| 1 | Recipient age ≥70 | 68 | 43 |
| 2 | Recipient age <46, donor age <43, | 87 | 79 |
| | recipient has private insurance as primary | | |
| | payment source at transplant | | |
| 3 | Recipient age <46, donor age <43, | 82 | 70 |
| | recipient has government insurance | | |
| | or self-pay as primary payment source at | | |
| | transplant | | |
| 4 | Recipient age <46, donor age >43, | 82 | 70 |
| | recipient HCV negative | | |
| 5 | Recipient age <46, donor age ≥43, | 71 | 54 |
| | recipient HCV positive | | |
| 6 | Recipient age <60 and ≥46, donor | 72 | 51 |
| | age ≥48, recipient diabetes >1 year | | |
| 7 | Recipient age >60 and <70, recipient | 75 | 58 |
| | without diabetes, recipient functional | | |
| | status at registration is between performing | | |
| | ADLs without assistance to full assistance | | |
| 8 | Recipient age >60 and <70, recipient | 70 | 49 |
| | diabetes >1 year, recipient functional status | | |
| | at registration is between performing | | |
| | ADLs without assistance to full assistance | | |
| 9 | Recipient age ≥60 and <70, recipient | 75 | 53 |
| | diabetes >1 year, recipient functional | | |
| | status at registration is between mostly in | | |
| | bed and fully active | | |
| 10 | Recipient age <60 and ≥46, donor age | 78 | 65 |
| | <48, recipient without diabetes, recipient | | |
| | functional status at registration is between | | |
| | performing ADLs without assistance to | | |
| | full assistance | | |
| 11 | Recipient age <60 and ≥46, donor age <48, | 71 | 54 |
| | recipient diabetes >1 year, recipient | | |
| | functional status at registration is disabled | | |
| | and moribund | | |
| 12 | Recipient age <60 and ≥46, donor age <48, | 79 | 63 |
| | recipient diabetes >1 year, recipient | | |
| | functional status at transplant is between | | |
| | 84without assistance and moribund | | |
| 13 | Recipient age <60 and ≥46, donor age ≥48, | 71 | 56 |
| | recipient without diabetes, recipient | | |
| | functional status at transplant is between | | |
| | without assistance and moribund | | |
| 14 | Recipient age >60 and <70, recipient | 76 | 59 |
| | without diabetes, recipient functional | | |
| | status at registration is between mostly in | | |
| | bed and fully active, no deceased donor | | |
| | pulmonary infection | | |
| 15 | Recipient age ≥60 and <70, recipient | 81 | 63 |
| | without diabetes, recipient functional | | |
| | status at registration is between mostly in | | |
| | bed and fully active, no deceased donor | | |
| | pulmonary infection | | |
| 16 | Recipient age <60 and ≥46, donor age ≥48, | 79 | 67 |
| | recipient without diabetes, recipient | | |
| | functional status at registration is between | | |
| | fully active and moribund, recipient HCV | | |
| | positive | | |
| 17 | Recipient age <60 and ≥46, donor age ≥48, | 84 | 60 |
| | recipient without diabetes, recipient | | |
| | functional status at transplant is between | | |
| | disabled and normal, recipient HCV | | |
| | negative | | |
| 18 | Recipient age <60 and ≥46, donor age ≥48, | 72 | 57 |
| | recipient without diabetes, recipient | | |
| | functional status at transplant is between | | |
| | disabled and normal, recipient HCV | | |
| | positive | | |
| 19 | Recipient age <60 and ≥46, donor age ≥48, | 84 | 72 |
| | recipient without diabetes, recipient | | |
| | functional status at registration is between | | |
| | fully active and moribund, recipient HCV | | |
| | negative, recipient functional status at | | |
| | transplant is between performing ADLs | | |
| | without assistance to requires | | |
| | considerable assistance | | |
| 20 | Recipient age <60 and ≥46, donor age ≥48, | 88 | 76 |
| | recipient without diabetes, recipient | | |
| | functional status at registration is between | | |
| | fully active and moribund, recipient HCV | | |
| | negative, recipient functional status at | | |
| | transplant is between requires occasional | | |
| | assistance and normal | | |

Determining the effect of covariate interactions of survival distribution traditionally requires prespecification of the functional form of the covariates, including their interactions. The SurvCART methodology uses the “conditional inference” framework to select the splitting variable via a parameter instability test and subsequently finds the optimal split. The result is an inimitable and flexible approach to determining covariate interaction. In this analysis, application of this technique resulted in a unique understanding of the covariate effect on survival probability. As the survival tree is constructed, branch points provide information on the effect of variable changes on long-term mortality. For example, liver transplant subgroups 17 and 18 differ by recipient hepatitis C status at the time of transplant, resulting in an estimated mortality probability difference of 12% and 13% at 5 and 10 years, respectively (Table 6). Determination of the degree to which variables affect survival could allow for targeted optimization to mitigate predicted long-term mortality (i.e., improving functional status, recipient treatment of hepatitis C prior to transplant). Currently, organ allocation is stratified by waitlist mortality probability without consideration for long-term patient outcome. While 1-year survival following liver transplant is excellent in the modern era, long-term patient-specific survival remains nuanced and is not currently included in organ allocation systems. Use of techniques that include long-term survival may assist in transplant decision-making or provide opportunities for intervention on modifiable factors, resulting in increased post-transplant survival.

In the foregoing specification, implementations of the disclosure have been described with reference to specific example implementations thereof. It will be evident that various modifications may be made thereto without departing from the broader spirit and scope of implementations of the disclosures as set forth in the following claims. The specification and drawings are, accordingly, to be regarding in an illustrative sense rather than a restrictive sense.

What is claimed is:

1. A method for predicting recipient survival after an organ transplant, the method comprising:
obtaining a trained pre-operative organ transplant machine learning model;
receiving a donor dataset corresponding to a plurality of factors relating to a given donor;
receiving a pre-operative recipient dataset corresponding to a plurality of pre-operative recipient factors relating to a given patient;
applying the donor dataset and the pre-operative recipient dataset to the pre-operative organ transplant machine learning model;
providing a result for the patient based on the trained pre-operative organ transplant machine learning model;
receiving a post-operative recipient dataset corresponding to a plurality of post-operative factors, the plurality of post-operative factors relating to a transplantation operation of the patient;
determining if the patient exhibits early graft failure;
applying the pre-operative recipient dataset and the post-operative recipient dataset to a post-operative organ transplant machine learning model; and
determining a survival probability for the patient.

2. The method of claim 1, wherein the plurality of factors relating to a given donor comprises:
a donor's age;
a donor's cytomegalovirus status; and
a donor's pulmonary infection status.

3. The method of claim 1, wherein the plurality of pre-operative recipient factors comprises:
a recipient's age;
a recipient's transplant history; and
a type of transplant procedure.

4. The method of claim 1, further comprising transmitting survival probability information to a physician, the survival probability information highlighting one or more factors that significantly influenced a subgroup categorization of a proposed organ transplant for the patient and at least one of: factors that can be altered prior to transplant surgery; factors that cannot be altered prior to transplant surgery; and factors that cannot be altered.

5. The method of claim 1, wherein the plurality of post-operative factors comprises:
a length of the patient's stay, the length of the patient's stay comprising an amount of days from transplant to discharge;
a patient's ventilator duration post-transplant; and
a patient's reintubation status post-transplant.

6. The method of claim 1, wherein the pre-operative organ transplant machine learning model utilizes a survival tree algorithm.

7. The method of claim 1, wherein the post-operative organ transplant machine learning model utilizes a survival tree algorithm.

8. The method of claim 1, wherein the pre-operative organ transplant machine learning model is trained using:
a recipient pre-operative training dataset, the recipient pre-operative training dataset corresponding to a plurality of organ recipients; and
a donor training dataset, the donor training dataset relating to a plurality of organ donors corresponding to the plurality of organ recipients.

9. The method of claim 8, wherein the post-operative organ transplant machine learning model is training using:
the recipient pre-operative training dataset;
the donor training dataset; and
a recipient post-operative training dataset.

10. A method for organ transplant prediction model training, comprising:
receiving a first training dataset relating to a plurality of organ recipients, the first training dataset comprising pre-operative and post-operative factors;
receiving a second training dataset relating to a plurality of organ donors, the plurality of organ donors corresponding to the plurality of organ recipients;
filtering the first training dataset to remove post-operative factors, data records for inapplicable recipient treatments, and data records for recipients with graft dysfunction to generate a recipient pre-operative training dataset;
training a pre-operative organ transplant machine learning model based on the recipient pre-operative training dataset and the second training dataset;
filtering the first training dataset to remove inapplicable recipient treatments, and recipients with graft dysfunction to generate a recipient post-operative training dataset; and
training a post-operative organ transplant machine learning model based on the recipient post-operative training dataset and the second training dataset, the post-operative machine learning model corresponding to the pre-operative machine learning model.

11. The method of claim 10, wherein the first training dataset comprises:
a plurality of recipient primary payment methods;
a plurality of recipient Hepatitis C statuses;
a plurality of recipient diabetes statuses; and
a plurality of recipient functional statuses before and after transplantation.

12. The method of claim 10, wherein the second training dataset comprises:
a plurality of donor ages;
a plurality of donor cytomegalovirus statuses; and
a plurality of donor pulmonary infection statuses.

13. The method of claim 10, wherein the pre-operative organ transplant machine learning model utilizes a survival tree algorithm.

14. The method of claim 10, wherein the post-operative organ transplant machine learning model utilizes a survival tree algorithm.

15. A system for recipient survival after organ transplant prediction, comprising:
a memory;
a processor communicatively coupled to the memory;
wherein the memory stores a set of instructions which, when executed by the processor, cause the processor to:
obtain a trained pre-operative organ transplant machine learning model;
receive a donor dataset corresponding to a plurality of factors relating to a given donor;
receive a pre-operative recipient dataset corresponding to a plurality of pre-operative recipient factors for a given patient;
apply the donor dataset and the pre-operative recipient dataset to the trained pre-operative organ transplant machine learning model;
provide a result for the patient based on the trained organ transplant machine learning model;
receive a post-operative recipient dataset corresponding to a plurality of post-operative factors, the plurality of post-operative factors relating to a transplantation operation of the patient;
determine if the patient exhibits graft dysfunction;
apply the pre-operative recipient dataset and the post-operative recipient dataset to a post-operative organ transplant machine learning model; and
determine a survival probability for the patient.
